(19) United States
(12) Patent Application Publication
(10) Pub. No.: US 2001/0046965 A1
(43) Pub. Date: Nov. 29, 2001

(54) Adenovirus E1-complementing cell lines

(76) Inventors: David Ayares, Lindenhurst, IL (US); Ramon Alemany, Grayslake, IL (US); Wei-Wei Zhang, Libertyville, IL (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/779,570

(22) Filed: Feb. 8, 2001

Related U.S. Application Data
(63) Continuation of application No. 08/810,039, filed on Mar. 4, 1997, now abandoned.
Application No. 08/810,039 is a continuation-in-part of application No. 08/658,961, filed on May 31, 1996.

(51) Int. Cl. A61K 48/00; C12N 7/01; C12N 5/06; C12N 15/861

(52) U.S. Cl. 514/44; 435/456; 435/235.1; 435/325

(57) ABSTRACT

A new series of helper cell lines for the complementation, amplification, and controlled attenuation of E1-deleted adenovirus are disclosed in the present invention. These cell lines are advantageous because they can complement adenovirus E1 gene deletions without production of replication competent adenovirus (RCA), thus making them safer for the large-scale production of adenovirus stock for use in human gene therapy trials. A preferred embodiment is an A549E1 cell line that contains only the Ad5 E1 DNA sequences sufficient for complementation of E1-deleted adenoviral vectors without sequences that overlap with the adenovirus vector.

In another aspect, the present invention embodies methods for the production of second generation A549-E1 complementing cell lines that, in addition to producing E1, also produce proteins required for further manipulation of adenoviral vectors. A preferred embodiment is an A549E1 cell line with DNA sequences that encode a polypeptide sufficient for packaging attenuation of E1-deleted helper virus, in order to enrich for packaging of mini-adenovirus.

### CROSS-REFERENCE

This application is a continuation-in-part of Ser. No. 08/658,961 filed May 31, 1996.

### FIELD OF THE INVENTION

This invention relates to novel cells and methods for use in propagating E1 deleted adenoviruses.

### BACKGROUND OF THE INVENTION

The majority of adenoviral vectors used in gene therapy applications were designed to have deletions in the E1 region of the adenovirus 5 (Ad5) genome. The E1 region, not including region IX, consists of 9% of the left end of Ad5 (1.2-9.8 map units), and is subdivided into two regions, E1A and E1B, each one coding for several proteins. Expression of E1A/E1B is required for virus replication and for expression of all other Ad5 proteins (E2-E4, Late Proteins; Ginsberg, H. S. The Adenoviruses. Plenum Press, New York. p.46-67(1984). Deletion of E1, therefore creates a replication-incompetent virus that, in theory, is silent for expression of all Ad5 proteins and expresses only the transgene of interest. Deletion of E1A and E1B is also of interest for safety reasons, since these two proteins, in combination, have been implicated in oncogenic transformation of mammalian cells (Graham, et al., In Cold Spring Harbor Symp. Quant. Biol. 39, p. 637-650(1974); Van Der Eb, et al., Gene 2, p.115-132 (1977); McKinnon, et al., Gene 19, p. 33-42(1982). All of the Class I adenovirus vectors used to date in human clinical trials, are deleted for E1.

E1 deficient adenoviral vectors are propagated in an Ad5 helper cell line called 293 (Graham, F. L. and Smiley, J, J. Gen. Virol. 36, p.54-72 (1977). 293 cells were derived by transforming human embryonic kidney cells with sheared fragments of Ad5 DNA. Genomic analysis revealed that 293 cells contain four to five copies per cell of the left 12% of the viral genome (including the entire E1 region) and approximately one copy per cell of 9% of the right end, the E4 region (Aiello, L., et al, Virology 94, p.460-469 (1979). While 293 cells are very efficient at producing high titers of E1-deficient adenovirus, they have the disadvantage that, due to the presence of Ad5 sequences besides E1 integrated into the 293 genome, recombination can occur with sequences in the E1-deficient adenovirus vector causing the production of E1-containing, replication-competent adenovirus (RCA). Depending on how early a passage the aberrant recombination event occurs during the amplification and propagation of the E1-deficient adenovirus, and which passage is used for large-scale production of the adenovirus stock, production of RCA in 293 cells can present severe ramifications for the safety of human gene therapy trials (Lochmuller, H., et al., Human Gene Therapy 5, p. 1485-1491 (1994). In addition to production of RCA, recombination in 293 cells can also cause deletions and rearrangements that affect transgene expression, thereby decreasing the titer of functional adenovirus particles. Recently, cell lines have been developed using defined Ad5 DNA fragments, including the E1 region; however these cell lines contain significant sequence overlap with homologous sequences in the E1-deleted adenovirus vectors, thus allowing for undesirable homologous recombination events and the possibility for generation of RCA (Fallaux, et al., Human Gene Therapy 7, p. 215-222 (1996); Imler, et al., Gene Therapy 3, p. 75-84 (1996).

In this invention, a series of cell lines have been generated containing only the minimal E1 gene region for the complementation of E1-deleted adenoviral vectors, in the absence of RCA.

### SUMMARY OF THE INVENTION

This invention encompasses a series of helper cell lines for the complementation, amplification, and controlled attenuation of E1-deleted adenovirus. These cell lines are advantageous because they can complement adenovirus E1 gene deletions without production of replication competent adenovirus (RCA). A preferred embodiment is an A549E1 cell line that contains only the Ad5 E1 DNA sequences sufficient for complementation of E1-deleted adenoviral vectors without sequences that overlap with the adenovirus vector. In a preferred embodiment, the E1 DNA sequences comprise E1A and E1B genes.

In another aspect, the present invention embodies methods for selectively propagating mini-adenovirus without generating RCA, by transfecting an A549E1 cell line with DNA sequences that encode a polypeptide sufficient for packaging attenuation of E1-deleted helper virus. In a preferred embodiment, the polypeptide comprises Cre recombinase. In another preferred embodiment, the polypeptide comprises TetR-KRAB.

### BRIEF DESCRIPTION OF THE DRAWINGS

FIG. 1 is a diagram indicating the structure of adenovirus sequences in a typical E1-deleted Ad helper virus (top line), in 293 cells (ref.5), and in other E1-containing cell lines, including 911 cells (ref.8) and A549E1-68 cells (this invention); and how recombination between homologous adenovirus sequences occurs to generate a replication-competent adenovirus (RCA).

FIG. 2 is a diagram of the CMV-E1 mammalian expression vector.

FIG. 3 GFP Expression and plaque formation in the E1-complementing cell line, A549E1-68, after infection with an E1-deleted adenovirus, Ad5-CA-GFP.

FIG. 4 is a Southern blot analysis of G418^{r} A549E1 clones.

FIG. 5 is a morphological comparison of parental A549 Cells and the E1-complementing cell line, A549E1-68.

FIG. 6A is a Western blot analysis of E1A protein expression in A549 cells, 293 Cells, and A549E1-68.

FIG. 6B is the metabolic ³⁵S labeling and immunoprecipitation of E1B proteins in A549 Cells, 293 Cells and 4 single-cell clones derived from A549E1-68.

FIG. 7 is a representation of the E1-deleted adenovirus vector, Ad5-CA-GFP.

FIG. 8 is an agarose gel analysis of 40 PCR reactions using E1A-specific primers for detection of RCA.

FIG. 9 is a diagram of a system for the attenuation of helper virus with a loxP-modified packaging signal.

FIG. 10 is a diagram of the pCMV-Cre-Puro vector.

FIG. 11 is a diagram of the pBS/loxP-stop/MCLpA vector.

FIG. 12 is a histochemical analysis of the expression of LacZ in A549E1-Cre puro^{R} clones after transient transfection with the pBS/loxP-stop/MCLpA test vector.

FIG. 13 is a bar graph depicting luciferase expression in both control cells (A549E1-68) and cell lines expressing the TetR-KRAB protein, following transient transfection with the pTetO7-CMV-L test vector.

### DETAILED DESCRIPTION OF THE INVENTION

This invention provides cell lines that can complement E1-deleted adenovirus without the disadvantage of undesirable recombination and RCA. These cell lines are obtained by cloning and expressing in the A549 cell line only those sequences that are required for E1 complementation and excluding from the cell line all other Ad5 sequences that have homology to the vector and could cause recombination to produce RCA.

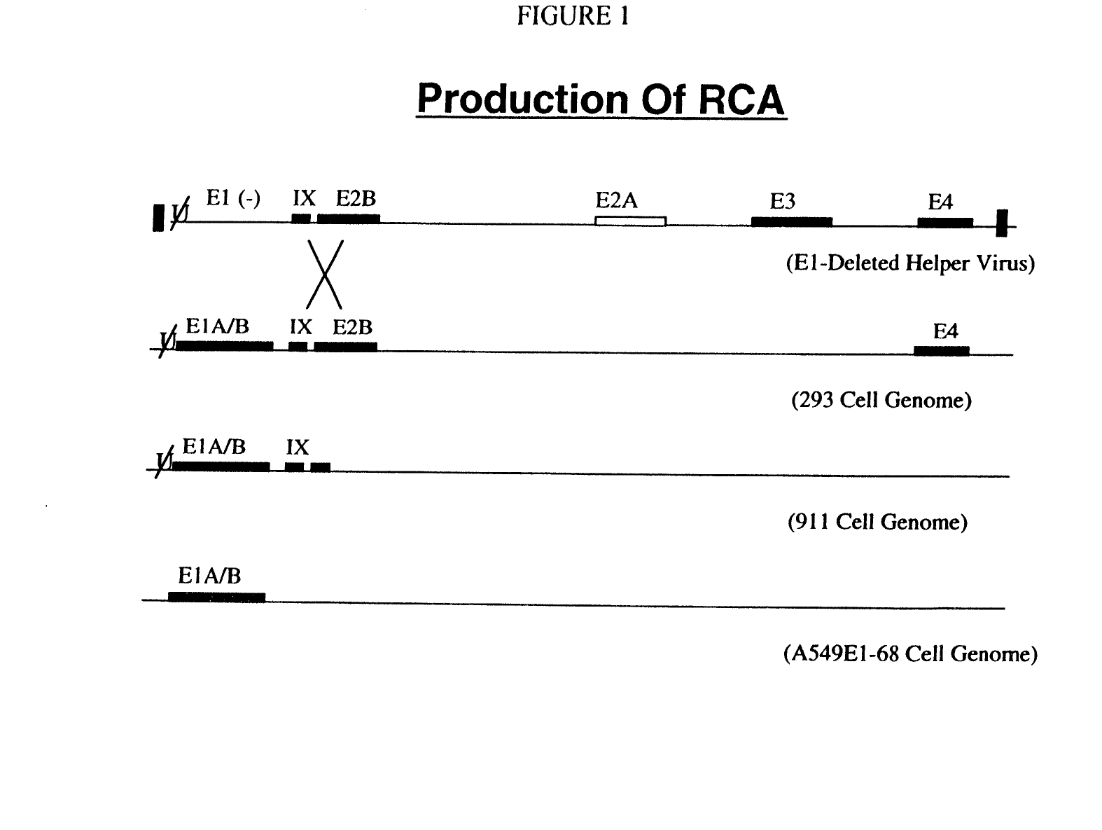

Currently 293 cells are used to propagate E1 deleted adenovirus. However, 293 cells harbor DNA fragments that make up 21% of the Ad5 genome. 293 cells therefore have significant sequence homology (outside of the E1 region) that overlaps with sequences in the adenoviral vectors, which can allow for homologous recombination events to produce a wild type adenoviral particle. FIG. 1 shows the structure of adenovirus sequences in 293 cells versus other E1-containing cell lines (including A549E1 cells), and how recombination between homologous adenovirus sequences occurs to generate a replication-competent adenovirus (RCA). Significant homology (more than 1000 base pairs) exists between Ad sequences 3′ to the E1 region in conventional adenovirus vectors, and sequences integrated into the helper cell genome of 293 cells and 911 cells. Reciprocal recombination across these homologous regions can result in the generation of a wild-type, E1(+), replication-competent adenovirus (RCA).

This invention embodies the cloning of an E1A and E1B-encoding DNA fragment from Ad5 into a mammalian expression vector. This E1 vector was stably transfected into human A549 cells to produce an E1 expression cell line. The genome of a representative cell line, A549E1-68, contains no sequence overlap with sequences present in the E1-deleted Ad helper virus and thus, recombination to produce RCA is not possible (FIG. 1). Characterization of this A549E1 cell line demonstrated the production of E1A and E1B proteins, high infectivity with adenovirus vectors, complementation of E1-deleted adenovirus to produce high-titer virus stocks, as well as, the lack of production of replication-competent adenovirus (RCA).

Further embodiments of the invention are described for the production of 2nd generation A549-E1 complementing cell lines which, in addition to producing E1, also produce proteins required for further manipulation of adenoviral vectors, thus providing a novel series of RCA-free adenovirus helper cell lines, as tools for novel virus production. The first example is an A549E1 cell line which expresses the Cre recombinase. This invention provides a novel E1-deleted helper virus whose packaging signal is flanked by loxP sites, and when this helper virus is propagated in an A549E1 cell line expressing Cre, the packaging signal is deleted by excision, thus attenuating helper virus packaging and enriching for packaging of mini-adenovirus (Ad5 virus which is devoid of all viral protein-coding sequences). This is advantageous because during the production of helper-dependent, mini-adenovirus it becomes necessary to attenuate helper virus packaging in order to enrich for the mini-virus. 293-Cre cells have been generated for this purpose (Parks, R., et al., P.N.A.S. 93, p.13565-13570 (1996), however, A549E1-Cre cells have an advantage in that they would perform this task in an RCA-free environment.

A further embodiment of the present invention includes an A549-E1 complementing cell line which expresses the TetR-KRAB fusion protein, which would be used to amplify, and control the packaging efficiency of an E1-deleted helper virus whose packaging signal has been modified to contain multiple tetracycline operator (tetO) sites. When this helper virus is propagated in an A549E1 cell line expressing TetR-KRAB, the repressor binds to the tetO sequence and specifically attenuates helper virus packaging, thus enriching for packaging of the mini-adenovirus which has a normal, wild-type packaging signal. Packaging of the helper virus can be restored by growing the cells and virus in the presence of tetracycline, which binds to the tet-KRAB repressor causing its dissociation from the tetO/packaging signal and a reversal of packaging repression. Detailed examples involving the derivation, characterization, and applications of these E1-complementing helper cell lines are described in the following sections.

### EXAMPLE 1

Construction of the E1A/E1B Vector

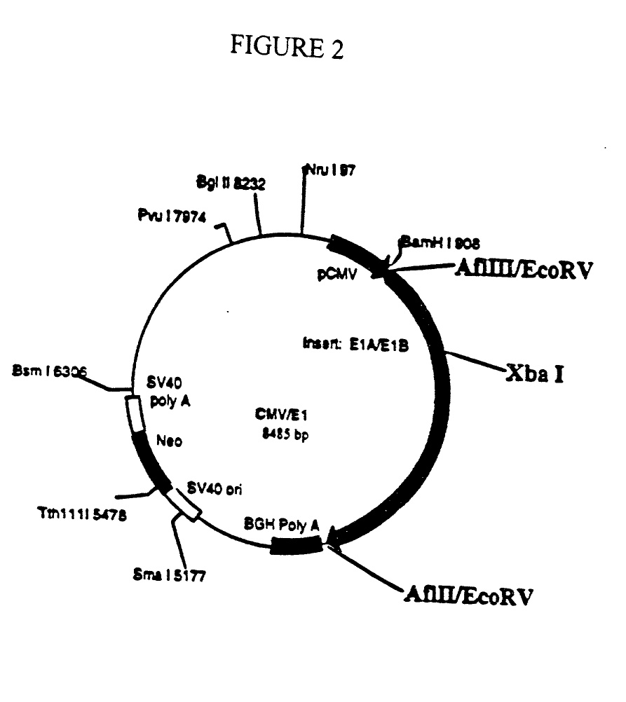

To generate an expression vector which harbors only the E1A/E1B sequences required for complementation, a 3.1 kb DNA fragment coding for Ad5 E1A and E1B genes was cloned in two pieces, sequentially, into the superlinker vector, pSL301 (Invitrogen). First, an 881 bp Afl III to XbaI fragment (Ad5 base pairs 462-1343) was cloned from pBRXad5KpnIC1 (a subclone of pJM17) into pSL301 (Afl III/XbaI). Second, a contiguous 2194 bp XbaI to Afl II (Ad5 base pairs 1343-3537) was cloned from pBRXad5XholC1 into the same vector. The resultant 3075 bp E1 fragment (in pSL301) contains the TATA box and RNA cap site for E1A, E1A coding sequence, complete E1B promoter, and E1B coding sequence, including the stop codon for E1B p55 protein, but not including region IX. The 3075 bp Afl III-Afl II E1A/E1B fragment (Ad5 base pairs 462-3537) was isolated, blunt-ended with Klenow enzyme, and blunt-end ligated into the EcoRV site of the mammalian expression vector, pCDNA3 (Invitrogen), under control of the CMV promoter/enhancer. This process generated an Ad5-E1 expression vector, pCMV-E1 (FIG. 2).

### EXAMPLE 2

Generation and Characterization of the E1 Cell Line

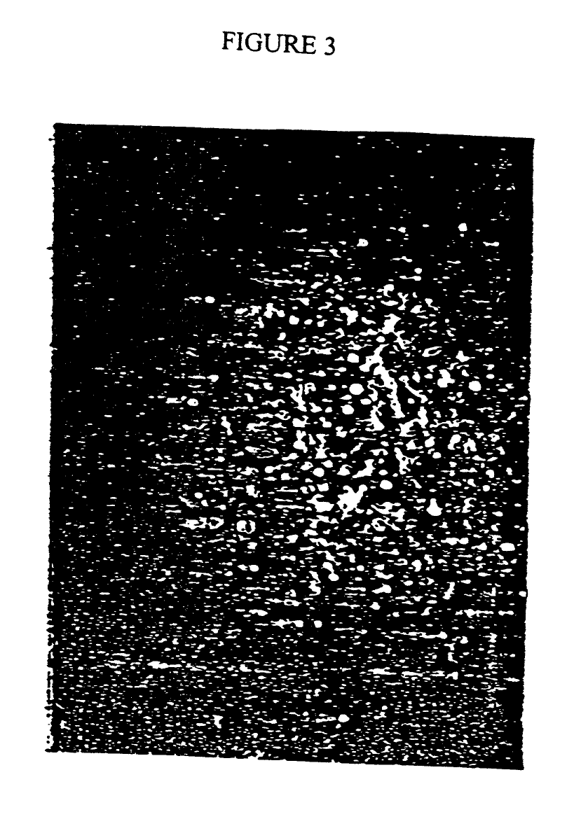

This CMV-E1 expression plasmid, (FIG. 2), was transfected using Lipofectamine (Gibco/BRL) into A549 human lung carcinoma cells (ATCC CRL 185) and G418^{R} colonies were isolated. Single-cell clones were screened for functional E1A/E1B expression. An E1-deleted adenovirus containing a green florescence protein (GFP) expression cassette, Ad5 CA-GFP, was used to infect the A549-E1 clones. Three days post-infection, clones were screened for production of E1-complemented Ad5 CA-GFP adenovirus by visual examination for cytopathic effect (CPE). One clone, A549E1-68, displayed 100% CPE in 3 days (FIG. 3), similar to that observed for 293 cells. The clear area in the center of the plaque is evidence of CPE caused by E1-complemented virus amplification. This clone also showed high infectivity, in that virtually 100% of the cells fluoresced green 24 hours post-infection (FIG. 3). The high infection rate and rapid generation of CPE induced in this cell line is strong evidence that functional E1A/E1B proteins are being produced that are capable of promoting replication and amplification of the E1-deleted Ad5-CA-GFP virus.

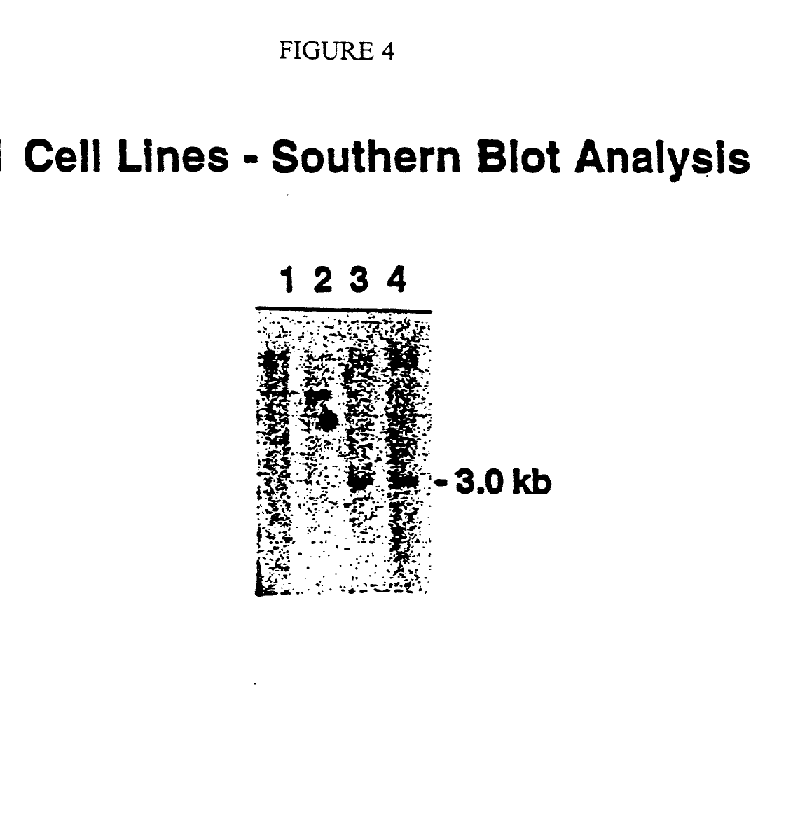

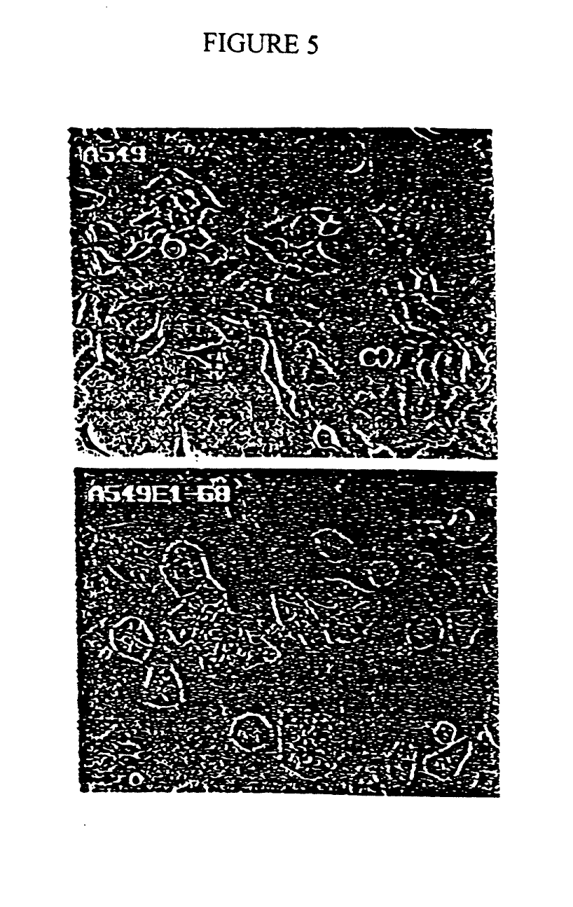

FIG. 4 shows a Southern blot using an E1 sequence-specific DNA probe. This assay demonstrated the presence of the CMV-E1 transgene in A549E1-68 (Lane 4), and a subclone of A549E1-68 (E1-68.3), but not in the parental A549 cell line (Lane 2). Sequences hybridizing with the E1-specific probe were also observed in 293 cells as expected since they complement E1-deleted adenovirus (Lane 3). FIG. 5 demonstrates that the morphology of the E1-transfected cells was significantly different from the parental A549 cell line. A549 cells at sub-confluent density (top panel) grow as distinct single cells with an elongated, fibroblast-like morphology, whereas the E1 cell line A549E1-68 (bottom panel) grows as colonies of cells with a more cuboidal morphology. A549E1-68 was also compared with 293 cells for production of E1-deleted adenovirus (Ad5 CA-GFP) by plaque assay and found to produce an equivalent titer of complemented virus (7×10⁹ pfu for A549E1-68 vs. 9×10⁹ pfu for 293).

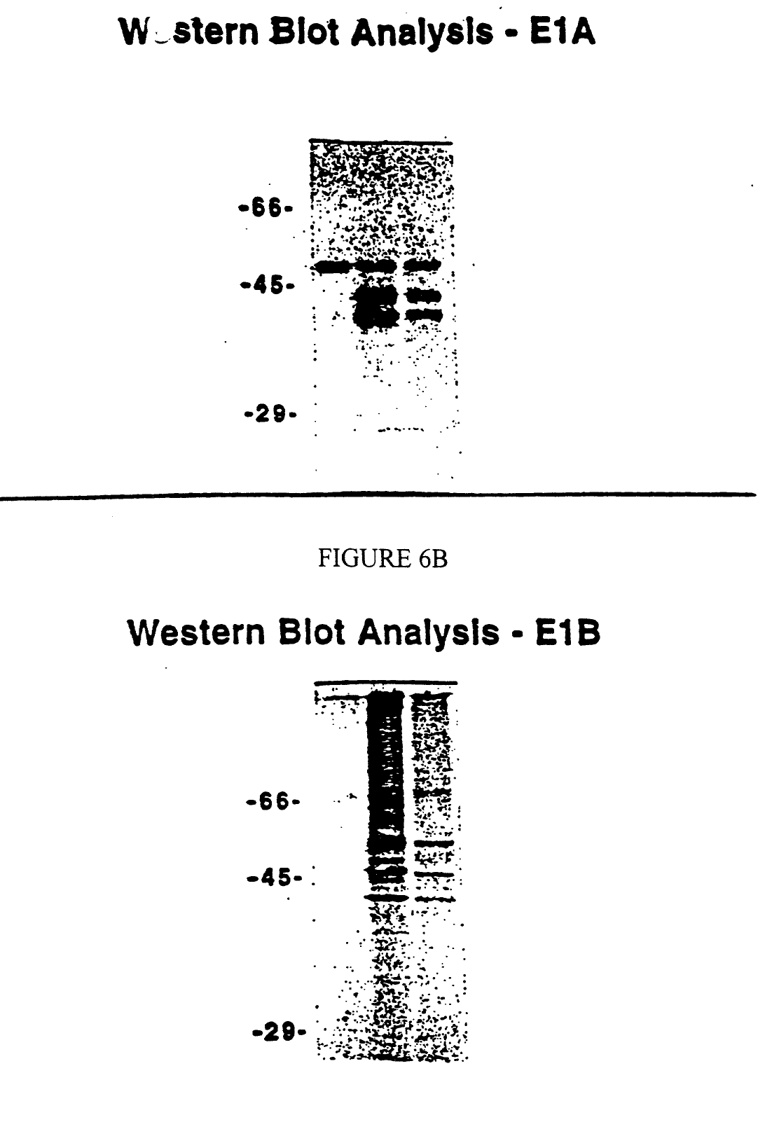

FIG. 6A shows a Western blot analysis using an E1A specific antibody (M73, Oncogene Science). This antibody detected two E1A-specific bands with apparent molecular weights of 46kd and 42kd in the A549E1-68 cell line (lane 3), corresponding to products expected from E1A 13S and 12S mRNAs (Ginsberg, 1984), and identical in size to those observed in 293 cells (lane 2). These E1A-specific bands were not detected in parental A549 cells (lane 1). FIG. 6B shows the immunoprecipitation of metabolically-radiolabeled proteins by a monoclonal antibody specific for E1B p55. A549E1-68 produced an immunoreactive band of approximately 55 kd (lane 3) that was not detected in parental A549 cells. This 55 kd, E1B-specific band, as well as secondary background bands, were observed in 293 cells also (lane 2). Extra “background” bands found in both experimental and control lanes have been observed by other authors and are attributed to co-immunoprecipitation of a variety of proteins including, cyclins, p53, and Rb. It is clear that A549E1-68 not only expresses E1A and E1B, but that they are functional, since this cell line can complement for production of high titer, E1 deleted, recombinant adenovirus.

### EXAMPLE 3

E1-deleted Adenovirus Produced in A549E1 Cells is RCA-free

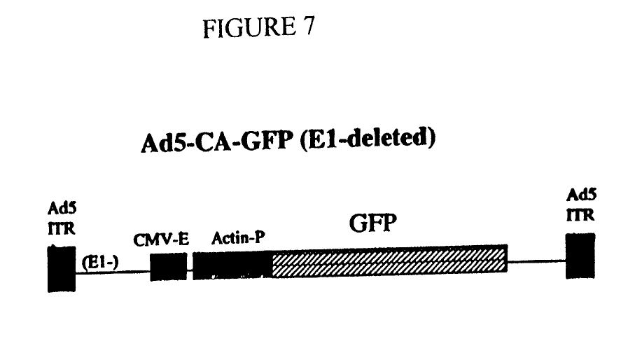

To prove that this new Ad5 helper cell line can complement without production of RCA, a series of PCR RCA assays were performed following amplification in A549E1 cells of the E1-deleted Ad5-CA-GFP adenovirus vector. The Ad5-CA-GFP vector is illustrated in FIG. 7. It contains a transcriptional control element consisting of the CMV enhancer and the β-actin promoter and the deletion of E1 sequences includes a lack of Ad5 DNA through base pair 3550. Since the E1A/E1B complementing region contained in the A549E1 cells extends only to base pair 3537, there is no overlapping sequence homology to allow RCA production. E1-deleted vectors with smaller deletions may still contain some E1 DNA and should be avoided, as they could still allow RCA to occur at a low frequency.

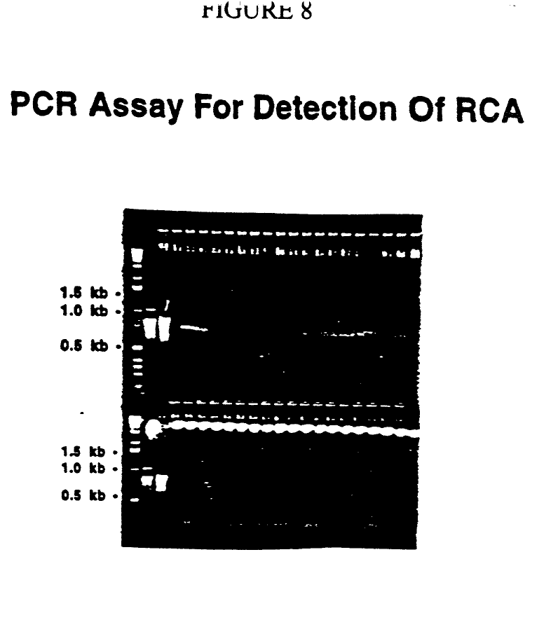

For the PCR RCA assay, Ad5-CA-GFP virus was serially propagated through 20 passages on A549E1-68 cells. Following serial propagation and virus amplification, Ad5-CA-GFP virus DNA was isolated by freeze-thaw lysis, and PCR was performed using primers specific for either the E1A region or the E2B region. Amplification of an 880 bp E2B product serves as a PCR positive control, while the presence of a 1086 bp E1A-specific product is evidence that an E1 (−) replication-competent adenovirus (RCA) has been produced during amplification of the E1 (−) Ad5-CA-GFP. 20 ug of Ad5-CA-GFP virus DNA (equivalent to 1×10¹⁰ virus particles), obtained from amplification in A549E1 cells, was divided into 40 PCR reactions and tested for RCA using the E1A primers (FIG. 8). For both top and bottom panels of FIG. 8, lane 1 contains 1 kb DNA markers, lane 2 contains wild type Ad5 virus DNA, lane 3 consists of PCR of Ad5CA-GFP virus DNA (E1-) isolated from 20+ passages on A549E1-68 cells using E1A and E2B specific primers (positive control), and lanes 4-20 consist of PCR of Ad5-CA-GFP virus DNA (E1-) isolated from 20+ passages on A549E1-68 cells, using E1A-specific primers only. No 1086 bp E1 region specific PCR fragments were detected in any of the reactions indicating that no RCA was present in the virus prep.

A second, CPE-based RCA assay was performed by amplifying E1-deleted adenovirus (Ad5-CA-GFP) on A549E1-68 cells and testing the amplified virus by passaging, on normal A549 cells (don't make E1) for production of E1-containing RCA. Plaque formation (CPE) on a monolayer of normal A549 cells would provide evidence for the production of wild-type (E1 +) virus during amplification on the E1 helper cell line, A549E1-68.2×10¹⁰ E1 (−) virus particles (amplified using A549E1-68) were used to infect each of five 150 mm plates of normal A549 cells (1×10¹¹ particles total). No CPE or single plaques were detected after 8 days on any of the A549 plates, indicating the absence of any E1 (+) RCA virus in the A549E1-68-amplified virus prep. Therefore, using two sensitive assays for detection of RCA, no wild-type recombinant E1 (+) virus was detected, supporting the utility of this cell line for the amplification and large-scale preparation of E1-deleted adenoviral vectors in the absence of RCA.

### EXAMPLE 4

Generation of an A549E1 Cre Cell Line

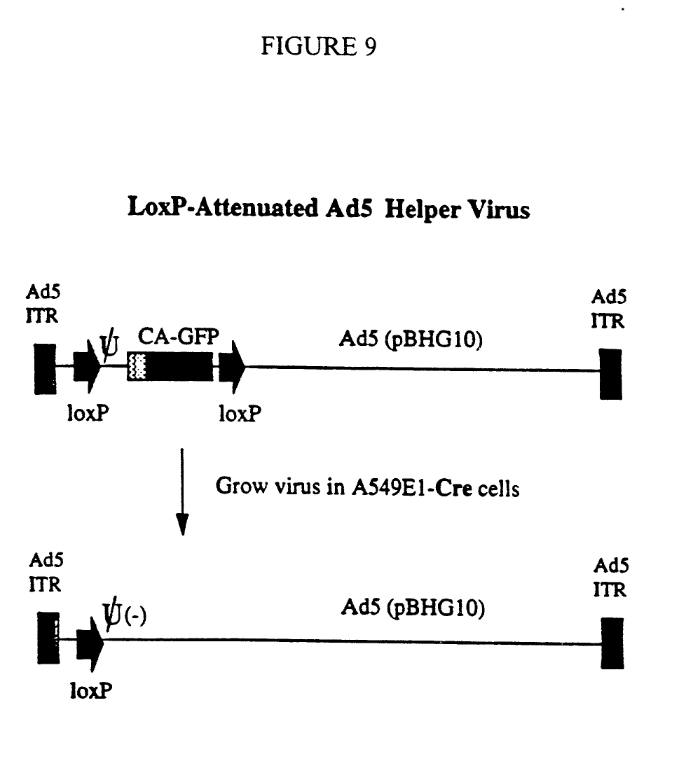

A 2nd generation E1-complementing cell line was generated using the A549E1-68.3 clonal line for transfection with Cre recombinase. This cell line will both complement E1-deleted adenovirus vectors and mediate the excision of sequences surrounded by loxP sites. Our primary use for this cell line is to further attenuate packaging of an Ad5 helper virus, whose packaging signal is flanked by two loxP sites (FIG. 9), in order to enrich for packaging of the desired E1 deficient, mini-adenovirus vector. 293 cells expressing the Cre recombinase were generated for a similar purpose by Parks et al. (P.N.A.S. 93:13565-13570), and were shown to increase the titer of E1-deleted vector virus 10 fold per passage, demonstrating the overall utility of this system for removal of helper virus. The A549E1-Cre cell line described in this invention will not only attenuate helper virus packaging in a similar fashion, it also has the advantage that any adenovirus produced will be free of deleterious RCA.

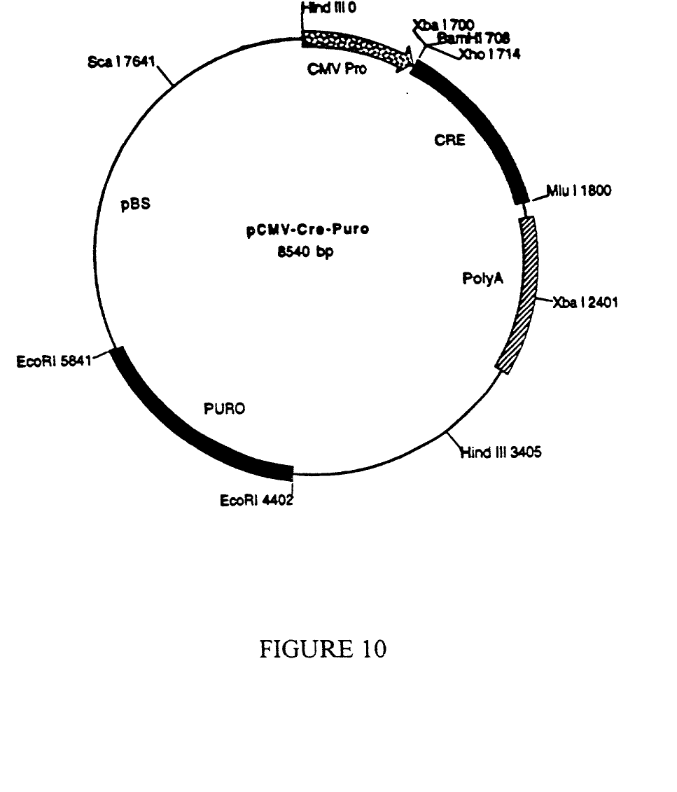

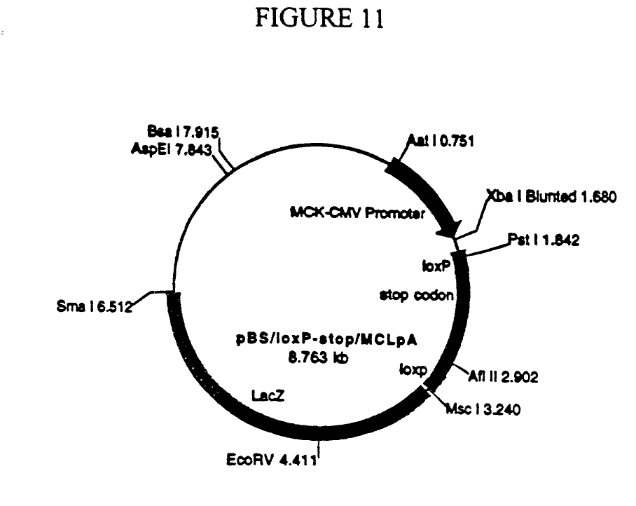

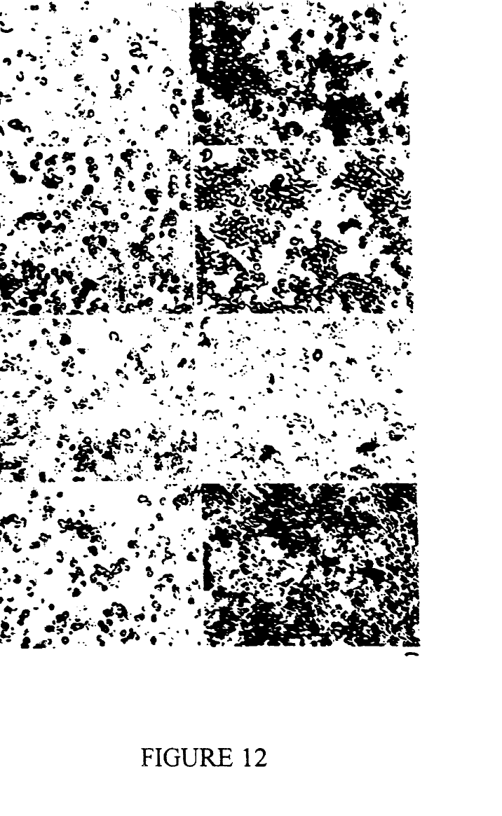

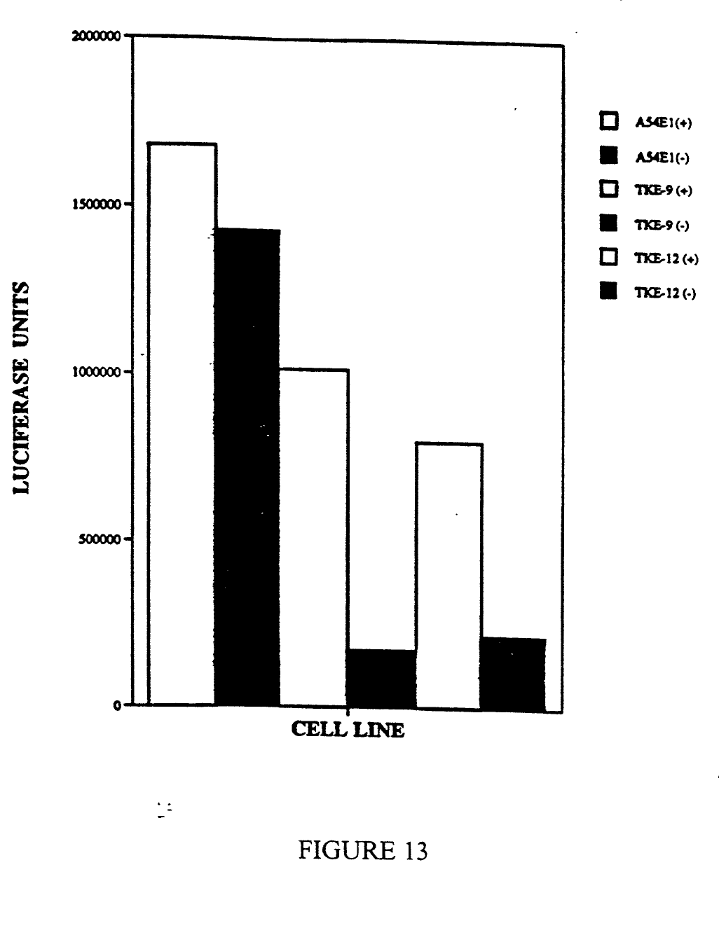

As a first step towards the production of the A549E1 Cre cell line, a Cre expression vector was constructed. A 1440 bp SV40 promoter-puromycin cassette (for selection in Neo^{R} A549E1 cells) was cloned into a unique EcoRI site of the CMV-Cre vector (pBS185, Gibco/BRL) to generate pCMV-Cre-Puro (FIG. 10). The pCMV-Cre-Puro vector was transfected by electroporation into A549E1-68 cells, and puromycin^{R} (“puro^{R}”) clones were isolated. These puro^{R} clones were then screened for expression of functional Cre recombinase. The plasmid pBS/loxP-stop/MCLpA contains a lacZ cassette that is non-functional due to the presence of a stop codon (FIG. 11). This stop codon is surrounded by loxP sites, such that the propagation of this vector in a cell line producing Cre would excise the stop signal and activate the lacZ gene. The pBS/loxP-stop/MCLpA vector was transiently transfected into each of the A549E1-Cre clones, and after 24 hours. the transfected cells were fixed and stained with X-Gal. FIG. 12 displays the results of these experiments. Panel A represents parental A549E1-68 cells (no Cre) while panels B-H represent lacZ expression in seven different puro^{r} A549E1-Cre clones. Expression of lacZ (due to expression of Cre) was observed as blue cells, at a frequency ranging from 1% to 50% in 20/26 puro^{R} clones. This range of LacZ-expressing cells is most likely a reflection of the transient transfection efficiency of the different puro^{R} clones with the pBS/loxPstop-MCLpA vector, although it could also reflect variations in Cre recombinase expression in different cell lines. Western blot analysis using an anti-Cre antibody (Pharmingen), confirmed the presence of the 35 kd Cre protein in these cell lines. Experiments to assess the attenuation of Ad helper virus containing a packaging signal flanked by two loxP sites are in progress.

### EXAMPLE 5

Generation of an A549E1 Cell Line Expressing TetR-KRAB

A highly conserved, 75 amino acid protein called the Kruppel-associated box (KRAB) was recently isolated by Margolin, et al. (P.N.A.S. 91, p.4509-4513 (1994). The KRAB box is a member of the Kox-1 family of human zinc finger proteins and was subsequently shown to be a strong transcriptional repressor. By fusing the KRAB domain from Kox-1 to the Tet repressor derived from Tn10 of Escherichia coli, a hybrid protein was generated, TetR-KRAB, which allows tetracycline-controlled silencing of eukaryotic promoters (Deuschle, et al., Mol. and Cell. Biol. 15 p. 1907-1914 (1995). The TetR-KRAB repressor binds to tetO sequences present in a transcriptional control region and represses transcription of genes placed as far as 3 kb downstream.

The present invention describes a system for tetracycline-controlled inhibition of helper virus packaging, comprising multiple tetO sequence in the helper virus packaging signal sequence, and an E1 helper cell line that constitutively expresses the TetR-KRAB protein. The helper virus is still capable of replicating and providing all the necessary proteins, in trans, required for replication of the miniAd vector, however, its packaging is attenuated due to binding of the TetR-KRAB protein to the tetO sites in the packaging signal. The overall goal is to hinder or repress helper virus packaging, thus enriching for vector virus packaging. This packaging repression is reversible, since in the presence of tetracycline, the TetR-KRAB repressor dissociates from the tetO sequences, and packaging is restored. Details of this tetO-controlled helper virus were presented in an earlier patent application (Ser. No. 08/658,961, filed May 31, 1996).

The TetR-KRAB expressing cell line was derived using the A549E1-68 helper cell line described in Example 2. A549E1-68 cells were transfected with a TetR-KRAB gene under control of the CMV promoter (see Deuschle et al., Mol. Cell. Biol. 15 p. 1907-1914 (1995). The TetR-KRAB vector also contains a hygromycin resistance gene for selection in mammalian cells. A test vector (see Deuschle, et al., Mol. and Cell. Biol. 15 p.1907-1914 (1995) that has a luciferase reporter gene under control of the CMV promoter fused to a TetO sequence was used for transient transfection into cells lacking a TetR-KRAB repressor. This transfection results in high level expression of luciferase, whereas transfection into A549E1 cells expressing the TetR-KRAB protein will result in the repression of luciferase due to binding of the repressor to tetO sites in the test vector. Hygromycin-resistant A549E1-TetR-KRAB clones were transfected with pTetO-CMV-L by electroporation and each clone was split into two wells of a 6-well plate. 24 hours post-transfection, cells from one duplicate well were refed with medium containing tetracycline, and the other duplicate well in medium without tetracycline. After another 24 hours, cells were lysed and assayed for luciferase expression using a Promega Luciferase Assay Kit. Two hygro^{R} A549E1 clones (TK#9 and TK#12) demonstrated a 4 to 6 fold repression of luciferase reporter activity when grown in the absence of tetracycline versus cells grown in media containing Tet, indicating expression of the TetR-KRAB repressor protein in the cells (FIG. 15). These A549E1-TetR-KRAB cell lines will be used to test attenuation of the TetO-controlled Ad helper virus.

These examples are intended to illustrate the present invention and are not intended to limit it in spirit or scope.

We claim:

1. A recombinant cell, comprising a mammalian cell capable of expressing a region of the adenoviral E1 sequence sufficient for complementation of E1-deleted adenoviral vectors without generating replication-competent adenovirus.

2. The recombinant cell of claim 1 further comprising said mammalian cell capable of expressing DNA sequences that encode a polypeptide sufficient for attenuation control of E1-deleted helper virus.

3. The recombinant cell of claim 2 wherein said DNA sequences comprise a Cre recombinase-encoding DNA.

4. The recombinant cell of claim 2, wherein said DNA sequences comprise a TetR-KRAB-encoding DNA.

5. A method for propagating E1-deleted adenovirus without generating replication-competent adenovirus comprising:
(a) transfecting a mammalian cell with a region of the adenoviral E1 sequence sufficient for complementation of said E1-deleted adenovirus without generating replication-competent adenovirus;
(b) infecting said mammalian cells with said E1-deleted adenovirus; and
(c) growing said mammalian cells under conditions suitable for lysis of said mammalian cells by said complementation of E1-deleted adenovirus.

6. A method of selectively propagating mini-adenovirus without generating replication-competent adenovirus comprising:
(a) transfecting a mammalian cell with a region of the adenoviral E1 sequence sufficient for complementation of said E1-deleted adenovirus without generating replication-competent adenovirus;
(b) further transfecting said mammalian cell with DNA sequences that encode a polypeptide sufficient for attenuation control of E1-deleted helper virus;
(c) co-infecting said mammalian cells with said E1-deleted helper virus, said E1-deleted helper virus containing a modified packaging signal that is controlled by said polypeptide;
(d) growing said mammalian cells under conditions sufficient for lysis of said mammalian cells by said complementation of E1-deleted adenovirus; and for said attenuation control of said E1-deleted helper virus.

7. The method of claim 6, wherein
(a) said DNA sequences that encode a polypeptide sufficient for attenuation control of E1-deleted helper virus comprise a Cre recombinase-encoding DNA; and
(b) said modified packaging signal that is controlled by said polypeptide sufficient for attenuation of E1-deleted helper virus comprises loxP sites adjacent to said packaging signal.

8. The method of claim 6, wherein
(a) said DNA sequences that encode a polypeptide sufficient for attenuation control of E1-deleted helper virus comprise a TetR-KRAB-encoding DNA; and
(b) said modified packaging signal that is controlled by said protein for attenuation of E1-deleted helper virus comprises a tetO DNA sequence in said modified packaging signal.

9. A method for making a recombinant cell comprising:
(a) transfecting a mammalian cell with a region of the adenoviral E1 sequence sufficient for complementation of E1-deleted adenoviral vectors without generating replication-competent adenovirus; and
(b) selecting for recombinant cells that express said region of the adenoviral E1 gene.
